(19) United States
(12) Patent Application Publication
(10) Pub. No.: US 2013/0183249 A1
(43) Pub. Date: Jul. 18, 2013

(54) Stimulus Sensitive Magnetic Nanocomposite Using Pyrene Polymer, and Contrast Medium Composition Containing the Nanocomposite

(71) Applicants: Seung Joo Haam, Seoul (KR); Jin Suck Suh, Seoul (KR); Yong Min Huh, Seoul (KR); Jae Moon Yang, Seoul (KR); Eun Kyung Lim, Seoul (KR)

(72) Inventors: Seung Joo Haam, Seoul (KR); Jin Suck Suh, Seoul (KR); Yong Min Huh, Seoul (KR); Jae Moon Yang, Seoul (KR); Eun Kyung Lim, Seoul (KR)

(73) Assignee: INDUSTRY ACADEMIC COOPERATION FOUNDATION YOUNSEI UNIVERSITY, SEOUL (KR)

(21) Appl. No.: 13/812,631

(22) PCT Filed: Dec. 27, 2010
(86) PCT No.: PCT/KR10/09345

§ 371 Date: Mar. 11, 2013

(30) Foreign Application Priority Data
Jul. 29, 2010 (KR) 10-2010-0073673

(51) Int. Cl. A61K 49/18; A61K 39/44

(52) U.S. Cl. CPC A61K 49/1824; A61K 39/44
USPC 424/9.322; 424/178.1

(57) ABSTRACT

Provided are a stimuli-sensitive magnetic nanocomposite using a pyrene-conjugated polymer and a method of preparing the same. Particularly, the stimuli-sensitive magnetic nanocomposite includes magnetic nanoparticles, an amphiphilic compound including at least one hydrophobic domain having a material having a pyrene structure and at least one hydrophilic domain and a pharmaceutically active ingredient. Here, the amphiphilic compound surrounds the pharmaceutically active ingredient, and the pharmaceutically active ingredient surrounds the magnetic nanoparticle and is chemically bound to a hydrophobic domain. Accordingly, the magnetic nanoparticles and the pharmaceutically active ingredient are stable in an aqueous solution, have excellent magnetic properties and a rapid drug release behavior due to a specific stimulus, and exhibit targeting according to a tissue-specific binding component. Thus, the stimuli-sensitive magnetic nanocomposite has target specificity and serves as a contrast composition or pharmaceutical composition.

### CROSS-REFERENCE TO RELATED APPLICATION

This application claims priority to and the benefit of Korean Patent Application No. 2010-0073673, filed Jul. 29, 2010, the disclosure of which is incorporated herein by reference in its entirety.

### BACKGROUND

1. Field of the Invention

The present invention relates to a stimuli-sensitive magnetic nanocomposite using a pyrene-conjugated polymer and a contrast composition including the same.

2. Discussion of Related Art

Nanotechnology is technology for adjusting and controlling materials on an atomic or molecular level, which is suitable for creating new materials or devices, and thus applied in various fields including electronics, materials, communications, mechanics, medicine and pharmaceuticals, agriculture, energy and environment.

The current nanotechnology is developing in various ways, which is classified into three major parts: First, technology for synthesizing new superfine nano materials and elements; second, technology for manufacturing a nano device having a certain function by combining or arranging nano-sized materials; and third, technology for applying nanotechnology to biotechnology, which is called nano-biology.

Particularly, in the field of nano-biology, magnetic nanoparticles are used in a wide range of applications including separation of bio materials, probes for diagnosing magnetic resonance imaging (MRI), biosensors including a giant magnetic resistance (GMR) sensor and a micro fluid sensor, drug/gene delivery, and magnetic hyperthermia.

Specifically, the magnetic nanoparticles may be used as a probe (contrast agent) for MRI. The magnetic nanoparticles have been widely used for resonance imaging diagnosis so far since they reduce a spin-spin relax time of hydrogen atoms in water molecules around nanoparticles and amplify MRI signals.

Moreover, the magnetic nanoparticles may serve as a probe material for a GMR sensor. As the magnetic nanoparticles are sensed and bound to biomolecules patterned on a surface of the GMR biosensor, a current signal of the GMR sensor is changed due to the magnetic particles, and thereby the biomolecules may be selectively detected [U.S. Pat. Nos. 6,452,763, 6,940,277 and 6,944,939; U.S. Patent Application Publication No. 2003/0133232].

Furthermore, the magnetic nanoparticles may be applied in separation of biomolecules. For example, when a cell expressing a specific bio marker is mixed with other cells, as a magnetic field is supplied in an external environment after the magnetic nanoparticles are selectively bound to the specific bio marker, the desired cell may be separated in a direction of the magnetic field [U.S. Pat. Nos. 4,554,088, 5,665,582 and 5,508,164; U.S. Patent Application Publication No. 2005/0215687]. The magnetic nanoparticles may also be applied in separation of various other biomolecules such as proteins, antigens, peptides, DNAs, RNAs and viruses, rather than the cells. In addition, the magnetic nanoparticles may be applied to a magnetic micro fluid sensor to separate and detect biomolecules. As a very fine channel is formed on a chip and then magnetic nanoparticles flow therethrough, biomolecules can be separated and detected in the micro-unit fluid system.

Meanwhile, the magnetic nanoparticles may also be used in in vivo treatment through drug or gene delivery. As a drug or gene is loaded on the magnetic nanoparticles by chemical bonding or adsorption to be transferred to a desired location using an external magnetic field and released to a specific part, a selective therapeutic effect may be achieved [U.S. Pat. No. 6,855,749].

As another example of the application of the magnetic nanoparticles to the in vivo treatment, hyperthermia using magnetic spin energy may be provided [U.S. Pat. Nos. 6,530,944 and 5,411,730]. When an alternating current having a radio frequency is supplied from an external environment, the magnetic nanoparticles emit heat through spin flipping. Here, when a temperature around the nanoparticles is 40° C. or more, cells die due to high heat, and thus infected cells may be selectively killed.

To be used in the above uses, the magnetic nanoparticles necessarily have excellent magnetic properties, stable delivery and in vivo dispersity, that is, in a water-soluble environment, and easy bindability with bio active materials. To satisfy these conditions, various techniques have been developed so far.

In U.S. Pat. No. 6,274,121, paramagnetic nanoparticles including a metal such as an iron oxide, and particularly, nanoparticles to which a tissue-specific binding material or an inorganic material including a binding site coupled with a diagnostically or pharmaceutically active material is attached on a surface of the nanoparticles are disclosed. In U.S. Pat. No. 6,638,494, paramagnetic nanoparticles including a metal such as an iron oxide, and a method of preventing agglomeration and precipitation of the nanoparticles under gravity or in a magnetic field by attaching a specific carboxylic acid on a surface of the nanoparticles are disclosed. As the specific carboxylic acid, an aliphatic dicarboxylic acid such as maleic acid, tartaric acid, glutaric acid or citric acid, or an aliphatic polydicarboxylic acid such as cyclohexane or tricarboxylic acid was used. In U.S. Patent Application Publication No. 2004/58457, functional nanoparticles circled in a monolayer are disclosed. Here, bifunctional peptides may be attached to the monolayer, and various biopolymers including DNA and RNA may be bound to the peptides. In U.K. Patent No. 223,127, a method of preparing a magnetic nanoparticle component including an operation of forming magnetic nanoparticles in a protein template, and particularly, a method of encapsulating apoferritin with magnetic nanoparticles is disclosed. In U.S. Patent Application Publication No. 2003/190,471, a method of forming nanoparticles using a manganese-zinc oxide in a bi-micellear vesicle, and nanoparticles having enhanced properties through thermal treatment of the prepared magnetic nanoparticles are provided. In U.S. Patent Application Publication No. 2005/130,167, a method of synthesizing water-soluble magnetic nanoparticles surrounded by 16-mercaptohexadecanoic acid and detecting viruses and mRNA in mice by intracellular magnetic labeling of the synthesized magnetic nanoparticles using a TAT peptide which is a transfection agent is disclosed. In Korean Patent Application No. 1998-0705262, particles including a super paramagnetic iron oxide core particle treated with starch coating and optional polyalkylene oxide coating and an MRI contrast agent including the same are disclosed.

However, the water-soluble nanoparticles prepared by the above-described methods have the following disadvantages. That is, the nanoparticles disclosed in the above-mentioned references are mainly synthesized in an aqueous solution. In this case, the nanoparticles are difficult to control in size, and thus have non-uniform size distribution. In addition, since the nanoparticles are synthesized at a low temperature, they have low crystallinity, and tend to form a non-stoichiometric compound. Therefore, the nanoparticles prepared by the above methods have problems of agglomeration in in vivo application due to low colloidal stability in an aqueous solution and many non-selective bindings.

During investigation of these magnetic nanoparticles, the present inventors found a magnetic nanocomposite, which includes magnetic nanoparticles, an amphiphilic compound having a fluorescent material (including pyrene), at least one hydrophobic domain and at least one hydrophilic domain, and a pharmaceutically active ingredient, which are bound to each other or encapsulated, and thus applied for a patent [Korean Patent Application No. 2008-89139]. However, it was found that pyrene and a drug are bound by physical association (interaction between a hydrophobic material and a hydrophobic part of the amphiphilic polymer), and thus there is no difference in drug release behavior between an acid condition and a neutral condition, that is, according to pH conditions [refer to FIG. 13]. That is, there is a concern that there may be a side effect that drugs may be released in both cancer cells or tissues having an acidic environment and normal cells or tissues having a neutral environment.

### SUMMARY OF THE INVENTION

For this reason, the inventors completed the invention by identifying a specific drug release behavior in an acid condition by combining pyrene with a drug by a chemical bond.

Therefore, the present invention is directed to providing a pyrene-based amphiphilic compound, a stimuli-sensitive magnetic nanocomposite using the same, which has stability in an aqueous solution, excellent magnetic properties such as s contrast effect for MRI and different drug release behavior according to stimuli, and a method of preparing the same.

The present invention is also directed to providing a contrast composition and pharmaceutical composition including the magnetic nanocomposite.

One aspect of the present invention provides a stimuli-sensitive magnetic nanocomposite, which includes a core containing at least one magnetic nanoparticle, and a shell containing an amphiphilic compound having at least one hydrophobic domain and at least one hydrophilic domain. Here, the hydrophobic domain includes a material including a pyrene structure to which a pharmaceutically active ingredient is chemically bound.

Another aspect of the present invention provides a method of preparing a stimuli-sensitive magnetic nanocomposite by mixing magnetic nanoparticles, an amphiphilic compound having at least one hydrophilic domain and at least one hydrophobic domain containing a material including a pyrene structure to which a pharmaceutically active ingredient is chemically bound, and a pharmaceutically active ingredient.

Still another aspect of the present invention provides a target-specific contrast composition and pharmaceutical composition for simultaneous diagnosis and treatment including the stimuli-sensitive magnetic nanocomposite as an effective ingredient.

### BRIEF DESCRIPTION OF THE DRAWINGS

The above and other objects, features and advantages of the present invention will become more apparent to those of ordinary skill in the art by describing in detail exemplary embodiments thereof with reference to the adhered drawings, in which:

FIG. 1 is a schematic diagram of a stimuli-sensitive magnetic nanocomposite according to the present invention;

FIG. 2 is a schematic diagram illustrating preparation and applications of a target-specific stimuli-sensitive magnetic nanocomposite according to an exemplary embodiment of the present invention;

FIG. 3 is a transmission electron microscope (TEM) image of an amphiphilic compound including a pyrene structure according to Preparation Example 2;

FIG. 4 shows (a) a schematic diagram illustrating preparation of the amphiphilic compound including a pyrene structure according to Preparation Example 2, (b) a FT-IR result, (c) a result obtained by nuclear magnetic resonance analysis; and (d) a fluorescence and absorbance graph of an amphiphilic compound and a hydrophobic pyrene;

FIG. 5 is a fluorescence graph according to a pH condition and concentration of the amphiphilic compound including a pyrene structure according to Preparation Example 2;

FIG. 6 shows (a) a result obtained by thermal gravity analysis, (b) a x-ray diffraction pattern, (c) a magnetic property, and (d) an electron microscope image of a stimuli-sensitive nanocomposite according to Example 1;

FIG. 7 shows (a) MR images and the change in relaxation (R2) according to concentration of the stimuli-sensitive nanocomposite according to Example 1 and (b) fluorescence and absorbance graphs;

FIG. 8 shows (a) results of a drug release test according to a pH condition of the stimuli-sensitive nanocomposite according to Example 1 and (b) a graph showing a drug release coefficient obtained by the drug release test according to a pH condition;

FIG. 9 shows (a) MR images showing a binding degree of target cancer cells according to a concentration of the target-specific stimuli-sensitive nanocomposite according to Example 2 and a graph showing the change in relaxation of the cells according to these images, (b) a flow cytometry graph activated by fluorescence to identify target specificity of the target-specific stimuli-sensitive magnetic nanocomposite according to Example 2 and (c) a bar graph in comparison with relative values;

FIG. 10 shows (a) fluorescence microscope images for comparatively analyzing cell images according to bindability of the target-specific stimuli-sensitive magnetic nanocomposite according to Example 2 to a target cancer cell, and (b) a graph showing results of a toxicity test for the target-specific stimuli-sensitive magnetic nanocomposite according to Example 2;

FIG. 11 shows (a) MR images of behaviors of tendency to remain in a nude mouse and (b) a graph showing the change in relaxation of the target-specific stimuli-sensitive magnetic nanocomposite according to Example 2;

FIG. 12 shows (a) MR images of results obtained by measuring a diagnostic ability using a nude mouse, (b) a graph showing the change in relaxation of the target-specific stimuli-sensitive magnetic nanocomposite according to Example 2, and (c) a graph showing results obtained by measuring a therapeutic ability of the target-specific stimuli-sensitive magnetic nanocomposite according to Example 2 using a nude mouse; and

FIG. 13 shows drug release test results for a magnetic nanocomposite in which pyrene and a drug are physically bound as described in Korean Patent Application No. 2008-89139.

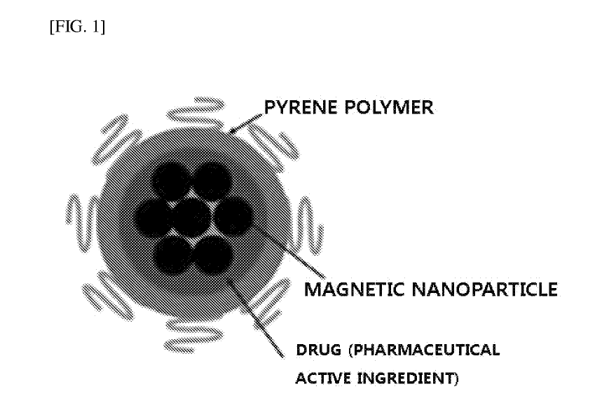

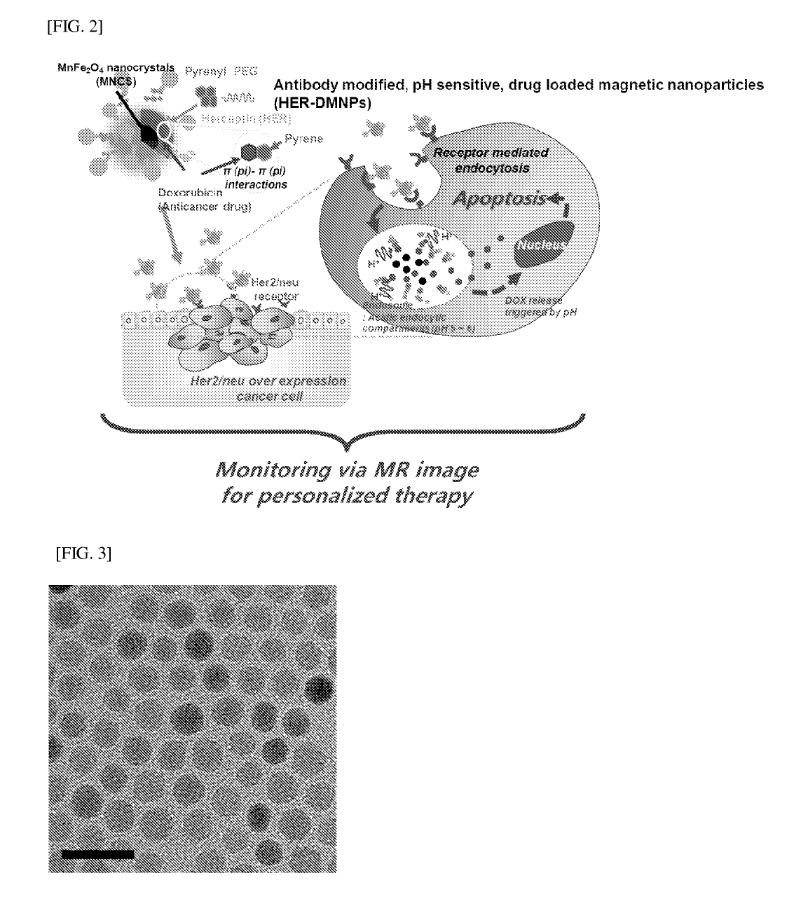

### DETAILED DESCRIPTION OF EXEMPLARY EMBODIMENTS

Hereinafter, exemplary embodiments of the present invention will be described in detail. However, the present invention is not limited to the embodiments disclosed below, but can be implemented in various forms. The following embodiments are described in order to enable those of ordinary skill in the related art to embody and practice the present invention.

Although the terms first, second, etc. may be used to describe various elements, these elements are not limited by these terms. These terms are only used to distinguish one element from another. For example, a first element could be termed a second element, and, similarly, a second element could be termed a first element, without departing from the scope of exemplary embodiments. The term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

The terminology used herein is for the purpose of describing particular embodiments only and is not intended to be limiting of exemplary embodiments. The singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, components and/or groups thereof, but do not preclude the presence or addition of one or more other features, integers, steps, operations, elements, components and/or groups thereof.

With reference to the appended drawings, exemplary embodiments of the present invention will be described in detail below. To aid in understanding the present invention, like numbers refer to like elements throughout the description of the figures, and the description of the same elements will be not reiterated.

Hereinafter, the configuration of the present invention will be described in detail.

The present invention relates to a stimuli-sensitive magnetic nanocomposite, which includes a core containing at least one magnetic nanoparticle, and a shell containing an amphiphilic compound having at least one hydrophobic domain and at least one hydrophilic domain. Here, the hydrophobic domain includes a material including a pyrene structure to which a pharmaceutically active ingredient is chemically bound.

In the stimuli-sensitive magnetic nanocomposite according to the present invention, the shell is formed by adding an amphiphilic compound to the core containing magnetic nanoparticles, the hydrophobic domain of the amphiphilic compound, which includes a pyrene structure, is bound to a pharmaceutically active ingredient, and the hydrophilic domain of the amphiphilic compound is distributed in an outermost region of the nanocomposite. Here, the hydrophobic domain of the amphiphilic compound is bound to a surface of the nanoparticle and a pharmaceutically active ingredient by chemical bonding such as a π-π interaction. Accordingly, the hydrophobic domain may serve to distribute nanoparticles into a matrix in the hydrophobic domain or bind to the surface of the nanoparticles, and control a drug release behavior due to stimuli by chemically binding a drug to one end of the hydrophobic domain when necessary. Meanwhile, the hydrophilic domain of the amphiphilic compound may be present in an outermost region of the nanocomposite, thereby stabilizing water-insoluble nanoparticles in a water-soluble medium and maximizing bioavailability.

The magnetic nanocomposite may have a diameter of 5 to 200 nm.

The magnetic nanoparticles have magnetism, and may have a diameter of, but not limited to, 1 to 1000 nm, and preferably 2 to 100 nm. Preferably, the nanoparticle may be a metal, magnetic material or magnetic alloy.

The metal may be, but is not particularly limited to, Pt, Pd, Ag, Cu or Au, which is used alone or in a combination of at least two thereof.

The magnetic material may be, but is not particularly limited to, Co, Mn, Fe, Ni, Gd, Mo, MM′₂O₄ or MₓO_{y} (M and M′ are each independently Co, Fe, Ni, Mn, Zn, Gd or Cr, and x and y satisfy “0<x≦3” and “0<y≦5”), which may be used alone or in a combination of at least two thereof.

The magnetic alloy may be, but is not particularly limited to, CoCu, CoPt, FePt, CoSm, NiFe or NiFeCo, which may be used alone or in a combination of at least two thereof.

In addition, the magnetic nanoparticles may be bound with an organic surface stabilizer to stabilize the binding with the amphiphilic compound. The binding of the organic surface stabilizer with the magnetic nanoparticles is formed by forming a complex compound by coordinating the organic surface stabilizer to a precursor material of the magnetic nanoparticle.

The organic surface stabilizer refers to an organic functional molecule capable of stabilizing a state and size of the nanoparticle, and for example, may be a surfactant.

The surfactant may be, but is not limited to, a cationic surfactant including an alkyl trimethylammonium halide, a neutral surfactant including a saturated or unsaturated fatty acid such as oleic acid, lauric acid or dodecylic acid, a trialkylphosphine or a trialkylphosphineoxide such as trioctylphosphine oxide (TOPO), trioctylphosphine (TOP) or tributylphosphine, an alkyl amine such as oleic amine, trioctylamine or octylamine, or an alkyl thiol, and an anionic surfactant including a sodium alkyl sulfate or a sodium alkyl phosphate. Particularly, in consideration of the stabilization and uniform size distribution of the nanoparticles, a saturated or unsaturated fatty acid and/or an alkyl amine is preferably used.

In addition, the amphiphilic compound may distribute nanoparticles in a matrix or may be bound to a surface of the nanoparticles, and chemically bond a pharmaceutically active ingredient to one end of the polymer.

The hydrophobic domain includes a hydrophobic compound to which a material including a pyrene structure is bound. The hydrophobic compound may be a saturated fatty acid, an unsaturated fatty acid or a hydrophobic polymer, which may be used alone or in combination of at least two thereof.

The saturated fatty acid may be, but is not particularly limited to, butyric acid, caproic acid, caprylic acid, capric acid, lauric acid (dodecylic acid), myristic acid, palmitic acid, stearic acid, eicosanoic acid or docosanoic acid, which may be used alone or in combination of at least two thereof.

The unsaturated fatty acid may be, but is not particularly limited to, oleic acid, linoleic acid, linolenic acid, arachidonic acid, eicosapentaenoic acid, docosahexaenoic acid or erucic acid, which may be used alone or in combination of at least two thereof.

The hydrophobic polymer may be, but is not particularly limited to, polyphosphazene, polylactide, polylactide-co-glycolide, polycaprolactone, polyanhydride, polymaleic acid or a derivative thereof, polyalkylcyanoacrylate, polyhydroxybutyrate, polycarbonate, polyorthoester, a hydrophobic polyamino acid or a hydrophobic vinly-based polymer, which may be used alone or in combination of at least two thereof.

The material including a pyrene structure may be, but is not particularly limited to, pyrene, pyrenebutyric acid, pyrene methylamine, 1-aminopyrene, pyrene-1-boronic acid or an organic molecule including a pyrene structure, which may be used alone or in combination of at least two thereof.

The hydrophilic domain may be a polyalkyleneglycol (PAG), polyetherimide (PEI), polyvinylpyrrolidone (PVP), hydrophilic polyamino acid (PAA), hydrophilic vinyl-based polymer, hydrophilic acryl-based polymer, or polysaccharide-based polymer such as dextran or hyaluronic acid, which may be used alone or in combination of at least two thereof.

The pharmaceutically active ingredient may be, but is not particularly limited to, an anticancer agent, an antibiotic, a hormone, a hormone antagonist, an interleukin, an interferon, a growth factor, a tumor necrosis factor, an endotoxin, a lymphotoxin, a urokinase, a streptokinase, a tissue plasminogen activator, a protease inhibitor, an alkylphosphocholine, a component marked with a radio isotope, a cardiovascular drug, a gastrointestinal drug or a neural drug, which may be used alone or in combination of at least two thereof.

Meanwhile, a pyrene-structured region included in the hydrophobic region of an amphiphilic polymer constituting the magnetic nanocomposite can be chemically bound with a structure of the pharmaceutically active ingredient, and thus a drug may be encapsulated with the magnetic nanocomposite.

The anticancer agent capable of being used in a treatment method according to the present invention may be, but is not limited to, epirubicin, docetaxel, gemcitabine, paclitaxel, cisplatin, carboplatin, taxol, procarbazine, cyclophosphamide, dactinomycin, daunorubicin, etoposide, tamoxifen, doxorubicin, mitomycin, bleomycin, plicomycin, transplatinum, vinblastin or methotrexate.

In addition, the magnetic nanocomposite according to the present invention may provide target specificity to the magnetic nanocomposite by introducing a tissue-specific binding component to the hydrophilic domain.

The tissue-specific binding component may be, but is not limited to, an antigen, an antibody, RNA, DNA, hapten, avidin, streptavidin, neutravidin, protein A, protein G, lectin, selectin, a radio isotope-marked component or a material capable of specifically binding to a tumor marker, which may be used alone or in combination of at least two thereof.

The nanocomposite of the present invention may be used to diagnose and/or treat various diseases relating to a tumor, for example, lung cancer, gastric cancer, breast cancer, ovarian cancer, liver cancer, bronchial cancer, nasopharyngeal cancer, larynx cancer, pancreatic cancer, bladder cancer, colon cancer and uterocervical cancer.

The tumor cell described above expresses and/or secretes a specific material that is rarely or never produced in normal cells, and is generally called a “tumor marker.” A nanocomposite prepared by bonding a material capable of specifically binding to the tumor marker to an active component binding region of the water-soluble nanoparticle may be useful in tumor diagnosis In the related art, various tumor markers and materials capable of being specifically bound thereto are disclosed.

In addition, in the present invention, the tumor marker may be classified into ligands, antigens, receptors and nucleic acids coding for them according to a mechanism of action.

TABLE 1

| Kinds | Example of Tumor Marker | Example of Active Ingredient |
|---|---|---|
| Ligand | C2 of Synaptotagmin I | Phosphatidylserine |
| Ligand | Annexin V | Phosphatidylserine |
| Ligand | Integrin | Integrin Receptor |
| Ligand | VEGF | VEGFR |
| Ligand | Angiopoietin 1, 2 | Tie2 Receptor |
| Ligand | Somatostatin | Somatostatin Receptor |
| Antigen | Carcinoembryonic Antigen | Herceptin (Genentech, USA) |
| Antigen | HER2/neu Antigen | Herceptin (Genentech, USA) |
| Antigen | Prostate-Specific Antigen | Rituxan (Genentech, USA) |
| Receptor | Folic Acid Receptor | Folic Acid |

When the tumor marker is a ligand, a material capable of specifically binding to the ligand may be introduced into an active ingredient of the nanocomposite according to the present invention, and a receptor or antibody capable of specifically binding to the ligand may be appropriate. The ligand available in the present invention and a receptor capable of specifically binding to the ligand may be, but is not limited to, C2 of synaptotagmin and phosphatidylserine, annexin V and phosphatidylserine, integrin and a receptor thereof, a vascular endothelial growth factor (VEGF) and a receptor thereof, angiopoietin and a Tie2 receptor, somatostatin and a receptor thereof, or a vasointestinal peptide and a receptor thereof.

When the tumor marker is an antigen, a material capable of specifically binding to the antigen may be introduced into an active ingredient of the nanocomposite according to the present invention, and an antibody capable of specifically binding to the antigen may be appropriate. The antigen available in the present invention and the antibody specifically binding to the antigen may be a carcinoembryonic antigen (colorectal cancer-labeled antigen) and Herceptin (Genentech, USA), a HER2/neu antigen (breast cancer-marked antigen) and Herceptin, or a prostate-specific membrane antigen (prostate cancer-labeled antigen) and Rituxan (IDCE/Genentech, USA).

As a representative example in which the tumor marker is a “receptor,” there is a folic acid receptor expressed in an ovarian cancer cell. A material capable of specifically binding to the receptor (folic acid in the case of the folic acid receptor) may be introduced as an active ingredient of the nanocomposite according to the present invention, and a ligand or antibody capable of specifically binding to the receptor may be appropriate.

As described above, the antibody is a very preferable active ingredient in the present invention. This is because the antibody selectively and stably binds to only a specific subject, and —NH₂ of lysine, —SH of cysteine, —COOH of asparaginic acid and glutamic acid, which is present in an Fc region of the antibody, is useful to bind to a functional group of an active ingredient binding region of the water-soluble nanocomposite.

Such an antibody may be commercially available or prepared according to a method known in the related art. Generally, the antibody may be prepared by immunizing a mammal (e.g., a mouse, rat, goat, rabbit, horse or sheep) with a suitable amount of antigens at least once. After a predetermined time, when a titer approaches a suitable level, antibodies are collected from a serum of the mammal. The collected antibody may be purified using a known process when necessary, and may be stocked in a frozen buffer. Details of the method are well known in the related art.

Meanwhile, the term “nucleic acid” includes RNA or DNA coding for the ligand, antigen or receptor, or a part thereof. Since nucleic acid, as shown in the related art, has a characteristic of forming base pairs between complementary sequences, a nucleic acid having a specific base sequence may be detected using a nucleic acid having a base sequence complementary with the base sequence. A nucleic acid having a base sequence complementary with the nucleic acid coding for the enzyme, ligand, antigen or receptor may be used as an active ingredient according to the present invention.

In addition, the nucleic acid may be useful to be bound with a functional group of an active ingredient binding region since it has functional groups such as —NH₂, —SH and —COOH at 5′- and 3′-ends.

Such a nucleic acid may be synthesized using an automatic DNA synthesizer (e.g., those available from Biosearch, Applied Biosystems, etc.) according to a standard method disclosed in the related art. For example, phosphothioate oligonucleotide may be synthesized by the method described in the literature [Stein et al. Nucl. Acids Res. 1988, vol. 16, p. 3209]. Methylphosphonate oligonucleotide may be prepared using an adjusted glass polymer support [Sarin et al. Proc. Natl. Acad. Sci. U.S.A. 1988, vol. 85, p. 7448].

The present invention also relates to a method of preparing a stimuli-sensitive magnetic nanocomposite, which includes: mixing magnetic nanoparticles, an amphiphilic compound including at least one hydrophilic domain and at least one hydrophobic domain having a pyrene structure to which a pharmaceutically active ingredient is chemically bound and a pharmaceutically active ingredient.

The method of preparing a stimuli-sensitive magnetic nanocomposite of the present invention will be described in detail by steps:

The magnetic nanoparticles may be prepared by a reaction of a precursor of the magnetic nanoparticle and an organic surface stabilizer in a solvent, and preferably, by mixing a precursor of the magnetic nanoparticle and an organic surface stabilizer in the presence of a solvent and thermally decomposing the resulting mixture.

First, a precursor of the nanoparticle is mixed into a solvent including a surface stabilizer.

Specific kinds of the magnetic nanoparticle and solvent are as follows:

As a precursor of the nanoparticle, a metal compound in which a metal binds to —CO, —NO, —O₅H₅, an alkoxide or another known ligand may be used. For example, various organic compounds including metal carbonyl based compounds such as iron pentacarbonyl (Fe(CO)₅), ferrocene, and manganese carbonyl (Mn₂(CO)₁₀); and metal acetylacetonate based compounds such as iron acetylacetonate (Fe(acac)₃) may be used.

In addition, the precursor of the nanoparticle may be a metal salt including a metal and a metal ion binding to a known anion such as Cl⁻ or NO₃⁻, for example, trichloroiron (FeCl₃), dichloroiron (FeCl₂) or iron nitrate (Fe(NO₃)₃).

Moreover, in the synthesis of an alloy nanoparticle and a composite nanoparticle, a mixture of at least two metals described above may be used.

In addition, the solvent may have a high boiling point approaching the thermal decomposition temperature of a complex compound in which the organic surface stabilizer is coordinated on a surface of the nanoparticle, and may be, for example, an ether compound such as octyl ether, butyl ether, hexyl ether, or decyl ether; a heterocyclic compound such as pyridine, or tetrahydrofuran (THF); an aromatic compound such as toluene, xylene, mesitylene, or benzene; a sulfoxide compound such as dimethylsulfoxide (DMSO); an amide compound such as dimethylformamide (DMF); an alcohol such as octyl alcohol or decanol; a hydrocarbon such as pentane, hexane, heptane, octane, decane, dodecane, tetradecane, or hexadecane; or water, which may be used alone or in combination of at least two thereof.

The mixing conditions are not particularly limited, and may be suitably adjusted according to the kinds of the precursor of the magnetic nanoparticle and surface stabilizer. The reaction may be performed at room temperature or less. Usually, it is preferable to perform heating and maintenance at 30 to 200° C.

After the mixing reaction, the precursor of the magnetic nanoparticle is thermally decomposed, thereby growing the nanoparticles.

Here, according to the reaction conditions, uniform sized and shaped metal nanoparticles may be prepared, and the thermal decomposition temperature may also be adjusted according to the kinds of the precursor of the nanoparticle and surface stabilizer. The reaction may be performed at 50 to 500° C.

The nanoparticles prepared as described above may be separated and purified by the known means.

The amphiphilic compound may be prepared by binding a hydrophilic compound and a hydrophobic compound having a pyrene structure using a crosslinking agent.

A specific kind of the hydrophilic compound, hydrophobic compound or material having a pyrene structure used in the above operations has been described above.

Here, the solvent used herein may be a polar aprotic-based organic solvent such as dimethylformamide, dioxane, tetrohydrofuran, pyridine or dimethylsulfoxide,

In the method of preparing a magnetic nanocomposite of the present invention, the magnetic nanocomposite may be prepared by a method using an emulsion.

According to the method using an emulsion, the magnetic nanocomposite is prepared by dissolving magnetic nanoparticles in an organic solvent to prepare a first oil phase, dissolving an amphiphilic compound and a pharmaceutically active ingredient in an organic solvent to prepare a second oil phase; mixing the first oil phase, the second oil phase and an aqueous phase to form an emulsion, and evaporating the oil phases from the emulsion.

As the organic solvent, a non-polar organic solvent such as chloroform, normal hexane, methylenechloride, toluene or benzene may be used.

Throughout the above operations, a stimuli-sensitive magnetic nanocomposite in which at least one magnetic nanoparticle has sensitivity due to a specific stimulus by coating of the amphiphilic compound may be prepared.

Particularly, a stimuli-sensitive magnetic nanocomposite in which the material having a pyrene structure having a binding region of the pharmaceutically active ingredient chemically binds to the pharmaceutically active ingredient is prepared. The chemical bond is a π-π interaction, which refers to a bond between electrons located in a π orbital filled with electrons when a double bond between chemical elements is formed.

In the method of preparing a magnetic nanocomposite of the present invention, an operation of binding a tissue-specific binding component to the magnetic nanocomposite may be further included, and here the tissue-specific binding component is chemically bound to a surface of the stimuli-sensitive magnetic nanocomposite using a crosslinking agent, thereby enhancing a cell targeting efficiency.

The operation of binding the tissue-specific binding component to a surface of the nanocomposite may include providing an active ingredient binding region to the hydrophilic compound using a crosslinking agent, and binding the active ingredient binding region to the tissue-specific binding component.

A specific kind of the tissue-specific binding component used in this operation has been described above.

In addition, the crosslinking agent may be, but is not particularly limited to, 1,4-diisothiocyanatobenzene, 1,4-phenylene diisocyanate, 1,6-diisocyanatohexane, 4-(4-maleimidophenyl)butyric acid N-hydroxysuccinimide ester, a phosgene solution, 4-(maleinimido)phenyl isocyanate, 1,6-hexanediamine, p-nitrophenyl chloroformate, N-hydroxysuccinimide, 1,3-dicyclohexylcarbodiimide, 1,1′-carbonyldiimidazole, 3-maleimidobenzoic acid N-hydroxysuccinimide ester, ethylenediamine, bis(4-nitrophenyl)carbonate, succinyl chloride, N-(3-dimethylaminopropyl)-N′-ethylcarbodiimide hydrochloride, N,N′-disuccinimidyl carbonate, N-succinimidyl 3-(2-pyridyldithio)propionate, or succinic anhydride, which may be used alone or in combination of at least two thereof.

The crosslinking agent is reacted with the amphiphilic compound and a part of the surface of the stimuli-sensitive magnetic nanocompo site to provide a binding region of the active ingredient such as —COOH, —CHO, —NH₂, —SH, —CONH₂, —PO₃H, —PO₄H, —SO₃H, —SO₄H, —OH, —NR₄⁺X⁻, -sulfonate, -nitrate, -phosphonate, a succinimidyl group, a maleimide group or an alkyl group.

In the operation, the binding of the active ingredient binding region of the surface of the stimuli-sensitive magnetic nanocomposite with an active ingredient of the tissue-specific binding component may be changed according to the kind of each active ingredient and its formula. Representative examples thereof are shown in Table 2.

TABLE 2

| I | II | III |
|---|---|---|
| R-NH₂ | R′-COOH | R-NHCO-R′ |
| R-SH | R′-SH | R-SS-R′ |
| R-OH | R′-(epoxy) | R-OCH₂C(OH)CH₂-R′ |
| RH—NH₂ | R′-(epoxy) | R-NHCH₂C(OH)CH₂-R′ |
| R-SH | R′-(epoxy) | R-SCH₂C(OH)CH₂-R′ |
| R-NH₂ | R′-COH | R-N═CH-R′ |
| R-NH₂ | R′-NCO | R-NHCONH-R′ |
| R-NH₂ | R′-NCS | R-NHCSNH-R′ |
| R-SH | R′-COCH₂ | R′-COCH₂S-R |
| R-SH | R′-O(C═O)X | R-OCH₂(C═O)O-R′ |
| R-(aziridine) | R′-SH | R-CH₂CH(NH₂)CH₂S-R′ |
| R-CH═CH₂ | R′-SH | R-CH₂CHS-R′ |
| R-OH | R′-NCO | R′-NHCOO-R |
| R-SH | R′-COCH₂X | R-SCH₂CO-R′ |
| R-NH₂ | R′-CON₃ | R-NHCO-R′ |
| R-COOH | R′-COOH | R-(C═O)O(C═O)-R′ + H₂O |
| R-SH | R′-X | R-S-R′ |
| R-NH₂ | R′CH₂C(NH²⁺)OCH₃ | R-NHC(NH²⁺)CH₂-R′ |
| R-OP(O²⁻)OH | R′-NH₂ | R-OP(O²⁻)—NH-R′ |
| R-CONHNH₂ | R′-COH | R-CONHNH═CH-R′ |
| R-NH₂ | R′-SH | R-NHCO(CH₂)₂SS-R′ |

I: functional group of active ingredient binding region
II: active ingredient
III: binding example according to reaction of I and II

The method of preparing a magnetic nanocomposite of the present invention may further include separating the magnetic nanocomposite generally produced in a precipitate by a conventional method, for example, centrifugation or filtration.

The present invention also relates to a contrast composition for simultaneous diagnosis and treatment, which contains the stimuli-sensitive magnetic nanocomposite as an effective ingredient.

The magnetic nanocomposite of the present invention may have sensitivities to both the magnetic nanoparticle and stimulus through the chemical binding with a pharmaceutically active ingredient or chemical encapsulation, and may be used as a delivery vehicle capable of diagnosing and treating a target part using MRI and optical imaging systems since the tissue-specific binding component is bound to a surface of the nanocomposite.

The contrast composition may use a carrier available for imaging as well as the magnetic nanocomposite, and the carrier available for imaging includes a carrier and vehicle conventionally used in the fields of medicine and pharmaceuticals. Specifically, the carrier may be, but is not limited to, an ion exchange resin, alumina, aluminum stearate, lecithin, a serum protein (e.g., human serum albumin), buffer materials (e.g., various phosphate, glycine, sorbic acid, potassium sorbate and partial glyceride mixtures of vegetable saturated fatty acids), water, a salt or an electrolyte (e.g., protamine sulfate, disodium hydrogen phosphate, potassium hydrogen phosphate, sodium chloride, and a zinc salt), colloidal silica, magnesium trisilicate, polyvinyl pyrrolidone, a cellulose-based substrate, polyethylene glycol, sodium carboxylmethylcellulose, polyacrlyate, wax, polyethylene glycol or lanoline.

In addition, the contrast composition of the present invention may further include a lubricant, a wetting agent, an emulsifier, a suspending agent, or a preservative, in addition to the above components.

In one aspect, the composition of the present invention may be prepared in a form of water soluble solution for parenteral administration. Preferably, Hank's solution, Ringer's solution or a buffer solution such as physically buffered saline may be used. To the water soluble suspension for injection may be added a substrate which increases the viscosity of suspension, such as sodium carboxymethylcellulose, sorbitol or dextran.

Another preferable aspect of the present composition may be in a form of sterile injectable formulation in an aqueous or oil suspension. Such suspension may be formulated using a suitable dispersing agent or wetting agent (e.g., Tween 80) and a suspending agent, according to the known technique in the related art.

In addition, the sterile injectable formulation may be a sterile injectable solution or suspension (e.g., a solution in 1,3-butanediol) in a non-toxic, parenterally acceptable diluent or solvent. The usable vehicle and solvent includes mannitol, water, Ringer's solution and an isotonic sodium chloride solution. In addition, a sterile non-volatile oil is usually used as a solvent or a suspending medium. For this purpose any less irritable non-volatile oil including synthetic mono or diglycerides may be used.

The contrast composition of the present invention may be used to sense a signal radiated from the stimuli-sensitive magnetic nanocomposite when administrated to tissues or cells separated from an object to be diagnosed and yield an image.

To sense the signal radiated by the stimuli-sensitive magnetic nanocomposite, an MRI apparatus may be used.

The MRI apparatus is a system for imaging signals transformed from the emitting energy of an atomic nucleus such as hydrogen through computer processing, in which the emitting energy is obtained by putting an organism in a powerful magnetic field, irradiating the organism with radio waves having a particular frequency, and stopping the radio waves after an atomic nucleus, such as hydrogen, present in a tissue of the organism absorbs energy and ends up in the upper energy state. The magnetic field or the radio waves do not interfere with bones. Thus, a clear three-dimensional tomographic imaging may be obtained longitudinally, transversely, and at an optional angle with regard to a tumor near bones, brain or bone marrow. In particular, the magnetic resonance imaging apparatus is preferably a T2 spin-spin relaxation magnetic resonance imaging apparatus.

The stimuli-sensitive magnetic nanocomposite of the present invention may be used for a nano probe and a drug or gene delivery vehicle for separation of biomolecules, diagnosis or treatment.

As a representative example of in vivo diagnosis using the stimuli-sensitive magnetic nanocomposite, molecular MRI diagnosis or a magnetic relaxation sensor may be used. The stimuli-sensitive magnetic nanocomposite shows a better T2 contrasting effect as its size is increased. Using such property, the magnetic nanocomposite may be used in a sensor for detecting biological moulecules. That is, particular biological molecules lead to aggregation of the magnetic nanocomposite, whereby the T2 magnetic resonance imaging effect is increased. The biological molecule is detected using this difference.

In addition, the magnetic nanocomposite according to the present invention can constitute a diagnosing material for a giant magnetic resistance (GMR) sensor. The magnetic nanocomposite may show a more excellent magnetic characteristic, better stability of colloid in a water solution and lower non-selective binding than conventional beads with a micrometer (10⁻⁶ m) size (U.S. Pat. Nos. 6,452,763, 6,940,277, 6,944,939, and U.S. Patent Application Publication No. 2003/0133232), and thus have the possibility to improve the detecting limit of the conventional GMR sensor.

The magnetic nanocomposite may also be used in separation and detection using magnetic micro fluid sensors, delivery of drugs or genes, and magnetic hyperthermia.

The present invention also relates a multi-diagnostic probe including the stimuli-sensitive magnetic nanocomposite and a diagnostic probe.

A T1 MRI diagnostic probe, an optical diagnostic probe, a CT diagnostic probe or a radio isotope may be used as the diagnostic probe.

For example, when a water soluble magnetic nanocomposite is combined with a T1 MRI diagnostic probe, simultaneous diagnosis for T2 MRI and T1 MRI can be performed. When the nanocomposite is combined with an optical diagnostic probe, MRI and optical imaging can be simultaneously performed. When the nanocomposite is combined with a CT diagnostic probe, MRI and CT diagnosis can be simultaneously performed. In addition, when the nanocomposite is combined with radioisotopes, MRI, PET and SPECT may be simultaneously performed.

The T1 MRI diagnostic probe includes a Gd compound or an Mn compound; the optical diagnostic probe includes an organic fluorescent dye, a quantum dot, or a dye labeled inorganic support (e.g., SiO₂, Al₂O₃); the CT diagnostic probe comprises an iodine (I) compound and gold nanoparticles; and the radioisotope includes In, Tc or F.

The present invention also relates to a pharmaceutical composition containing the stimuli-sensitive magnetic nanocomposite as an effective ingredient.

The pharmaceutical composition according to the present invention may be formulated in an oral dosage form such as granules, powders, liquids, tablets, capsules or dry syrups or in a parenteral formulation such as an injectable, but the present invention is not limited thereto.

In addition, the pharmaceutical composition according to the present invention may further include a pharmaceutically available carrier, and as the carrier, a conventional excipient, a disintegrating agent, a binding agent or a lubricating agent may be selectively used. For example, as an excipient, microcrystalline cellulose, lactose or low substituted hydroxycellulose may be used, as a disintegrating agent, sodium starch glycolate or anhydrous dibasic potassium phosphate may be used. As a binding agent, polyvinylpyrrolidone, low substituted hydroxypropylcellulose or hydroxycellulose may be used, and as a lubricating agent, magnesium stearate, silicon dioxide or talc may be selectively used.

Hereinafter, the present invention will be described with reference to Examples in detail. However, Examples are merely provided to explain the present invention, not to limit the present invention.

### PREPARATION EXAMPLE 1

### Preparation of Highly Sensitive Magnetic Nanoparticle Using Saturated Fatty Acid

7 nm magnetite (MnFe₂O₄) was synthesized by heating 2 mmol of iron triacetylacetonate (Aldrich) and 1 mmol of manganese triacetylacetonate (Aldrich) in 20 ml of a benzylether solvent including 0.6 mol of each of dodecylic acid and dodecyl amine at 215° C. for 2 hours and thermally decomposing the resulting mixture at 315° C. for 1 hour.

12 nm magnetite nanoparticles were prepared by heating 20 ml of a benzylether solution including 0.2 mol of dodecylic acid, 0.1 mol of dodecyl amine, 10 mg/ml of the 7 nm magnetite nanoparticles, 2 mmol of triacetylacetonate iron and 1 mmol of manganese triacetylacetonate at 115° C. for 30 minutes, at 215° C. for 2 hours and at 315° C. for 1 hour.

A transmission electron microscope image of the prepared magnetic nanoparticles is shown in FIG. 3.

### PREPARATION EXAMPLE 2

### Synthesis of Amphiphilic Compound Including Material Having Pyrene Structure

To synthesize an amphiphilic polymer (pyrenyl PEG) including a material having a pyrene structure, 0.001 mol of polyethyleneglycol (NH₂-PEG-COOH, Mw: 5,000 Da) having a hydrophilic hetero functional group and 0.003 mol of 1-pyrenebutyric acid N-hydroxysuccinimide ester (Py-NHS, Mw: 385.41 Da) as a stimuli-sensitive material were dissolved in 15 ml of dimethylformamide, and 0.2 ml of triethyleneamine was added, and the resulting mixture was reacted in a nitrogen atmosphere at room temperature for 48 hours. The synthesized pyrenyl PEG was precipitated in an excessive amount of ether to purify.

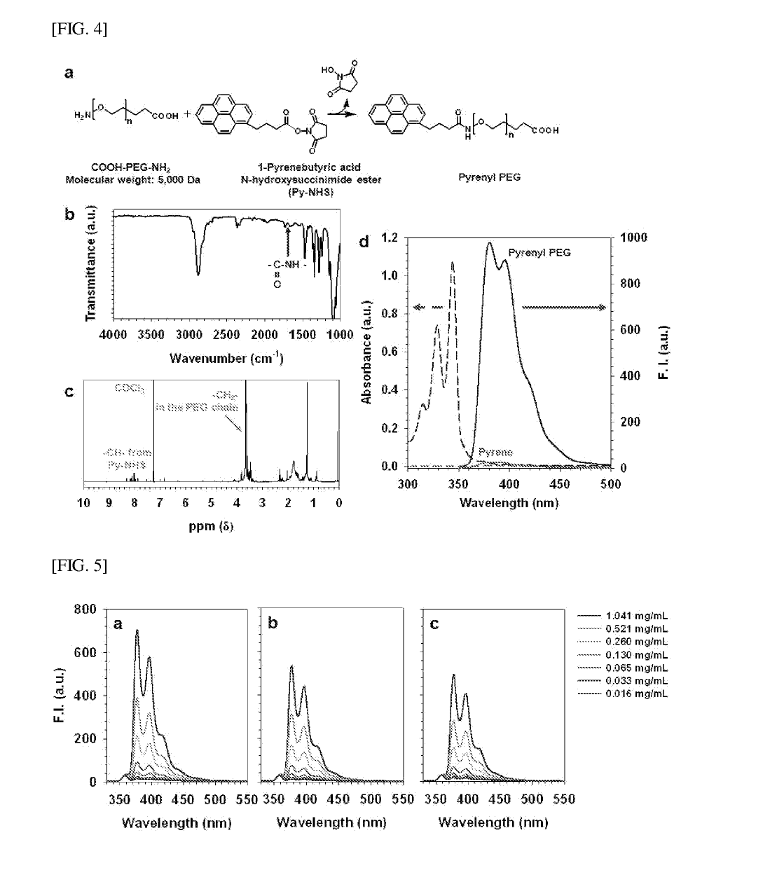

The synthesis process is shown in FIG. 4(A). An amide bond of the prepared pyrenyl PEG was identified by FT-IR at 1,612 cm⁻¹, and the result is shown in FIG. 4(B). Polyethyleneglycol having a hetero functional group was identified around 3.65 ppm (NMR) and peaks of 1-pyrenebutyric acid N-hydroxysuccinimide were identified around 7.82 and 8.12 ppm, and the results are shown in FIG. 4(C). A solubility of an aqueous phase of the pyrenyl PEG was measured using a fluorescence spectrometer, and compared with the solubility of an aqueous phase of pure pyrene to confirm that the stimuli-sensitive pyrene structure binds to the PEG, which is shown in FIG. 4(D). Particularly, high fluorescence caused due to interference of the formation of micelles by a hydrogen ion based on the same concentration at pH 5.5 was identified by measuring a difference in fluorescence of the pyrenyl PEG according to various pH conditions (pH 5.5, 7.4 and 9.8) and concentrations, and the results are shown in FIG. 5.

### EXAMPLE 1

### Preparation of Emulsion-type Stimuli-Sensitive Magnetic Nanocomposite

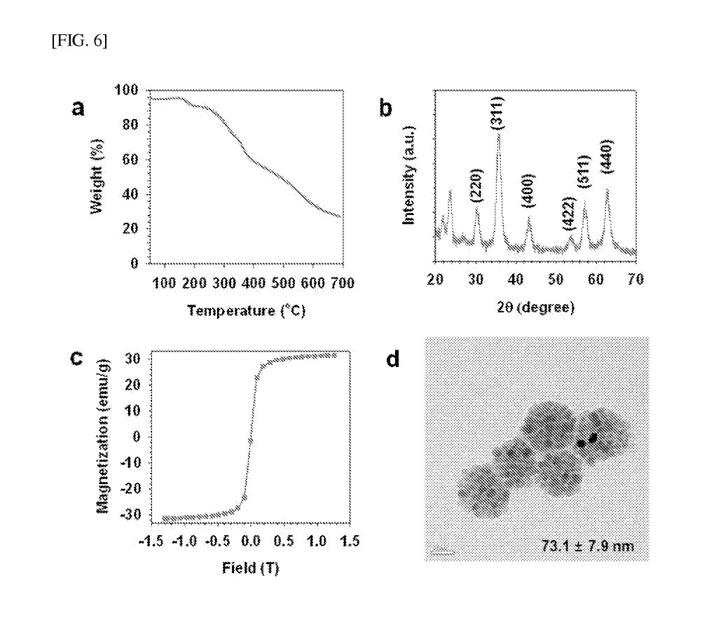

To form a π-π interaction, first, 100 g of the amphiphilic compound including a pyrene structure prepared in Preparation Example 2 and 5 mg of doxorubicin were dissolved in 4 ml of oil-phase chloroform, and 10 mg of the magnetic nanoparticles prepared in Preparation Example 1, and 10 mg of the magnetic nanoparticles prepared in Preparation Example 1 was dissolved in 1 ml of n-hexane. These two oil phases were mixed with 20 ml of aqueous phase to form an emulsion, and the emulsion was stirred for 12 hours to evaluate the oil phases. The resulting solution was centrifuged at 20,000 rpm for 45 minutes, and thus a stimuli-sensitive magnetic nanocomposite (73.1 ±7.9 nm) from which impurities were removed was obtained. The prepared particles were redispersed in 10 ml of PBS solution (pH 7.4), and the size and zeta charge were identified using a dynamic laser scattering method. An encapsulating ratio of the magnetic nanoparticles and crystallinity conservation were identified by thermal gravity analysis (TGA) and x-ray diffraction (XRD), and the results are shown in FIGS. 6(A) and (B). Supermagnetic properties and a shape of the composite were identified using a vibration sample magnetometer (VSM) by a transmission electron microscope, and the results are shown in FIGS. 6(C) and (D).

### EXPERIMENTAL EXAMPLE 1

### Analysis of MR Contrast Effect of Stimuli-Sensitive Magnetic Nanocomposite and Identification of Drug Encapsulation

To identify availability of the stimuli-sensitive magnetic nanocomposite prepared in Example 1 as an MRI contrast agent, an MR contrast effect of the magnetic nanocomposite was examined through measurement of r2 (T2 relaxivity coefficients).

Specifically, an MRI test was performed using a 1.5 T clinical MRI apparatus (Intera, Philips Medical System) having a micro-47 surface coil. An r2 (T2 relaxivity coefficients with unit of mM⁻¹s⁻¹) value of the magnetic nanocomposite was measured using a Carr-Purcell-Meiboom-Gill (CPMG) sequence at room temperature (TR=10 s, 32 echoes with 12 ms even echo space, number of acquisitions=1, point resolution of 156×156 μm, section thickness of 0.6 mm).

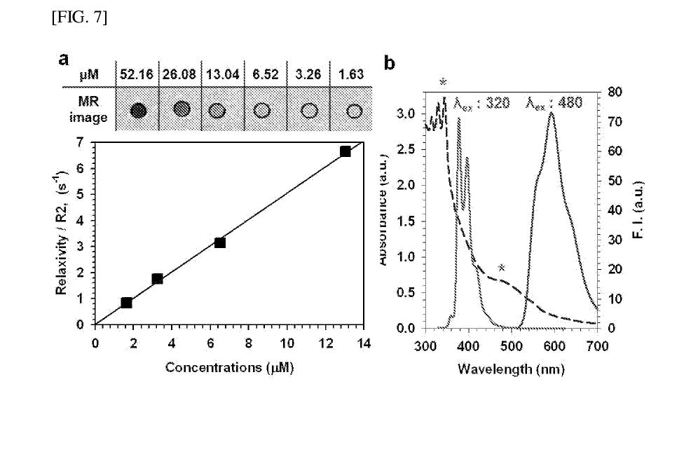

As shown in FIG. 7(A), r2 was clearly increased at 1.5 T as the concentration of the stimuli-sensitive magnetic nanocomposite was increased. A high r2 value was obtained by a clustering effect of the magnetic nanoparticles. In addition, as the concentration was increased, a significantly dark MR contrast was provided. Accordingly, it could be identified that the stimuli-sensitive magnetic nanocomposite, as an MRI contrast agent, ensured sufficient magnetic properties for molecular imaging. In addition, drug encapsulation in the composition was identified by absorbance and fluorescence spectrometers for the composite dispersed in a water-soluble phase, and the results are shown in FIG. 7(B). According to the absorbance at 480 nm and fluorescence of 600 nm, which are the original characteristics of doxorubicin, encapsulation was identified.

### EXPERIMENTAL EXAMPLE 2

### Drug Release Test of Stimuli-Sensitive Magnetic Nanocomposite

Stimuli sensitivity of the stimuli-sensitive magnetic nanocomposite prepared in Example 1 was identified by a drug release test.

First, after a titer curve according to the concentration of a drug was plotted by a

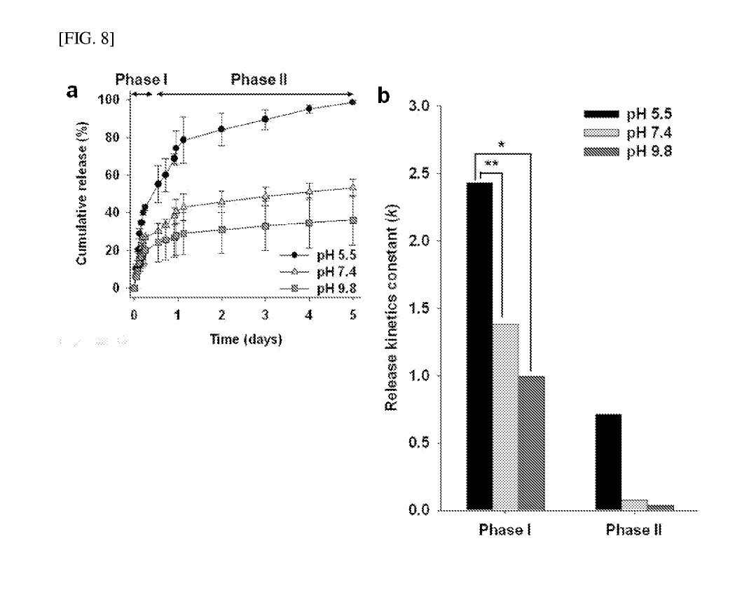

UV/visible spectrophotometer, the prepared stimuli-sensitive magnetic nanocomposite was dispersed in a buffer adjusted to each of pH 5.5, 7.4 and 9.8, and a concentration was measured by extracting a sample at regular time intervals. The results are shown in FIG. 8(A). Particularly, the time was divided into two sections, such as a first section of 0 to 0.5 days as phase I and a second section of 0.5 to 5 days as phase II to calculate a drug release coefficient (k) by Xᵢ=1−X_{inf}e−k (Xt: a drug release rate according to time, X_{inf}: the maximum efficiency of releasing the drug), and the results are shown in FIG. 8(B).

From the initial Phase I under an acid condition of pH 5.5, specific characteristics of the stimuli-sensitive magnetic nanocomposite shown under the acid condition (low pH) were identified through faster drug release behavior than other pH conditions.

Generally, since cancer cells or tissues have a more acidic environment condition than normal cells or tissues, when a fast drug release behavior was shown under an acid condition as described above, the drug was delivered but it had a low chance of being released in the normal tissues, thereby reducing a side effect. That is, since the drug was selectively released to a target cell, which was a cancer cell, the cell was possible to treat without a side effect.

### EXAMPLE 2

### Binding of Target-Specific Ligand to Stimuli-Sensitive Magnetic Nanocomposite

0.01 mmol of N-hydroxysuccinimide and 0.01 mmol of 1-ethyl-3-(3-dimethylaminopropyl)-carbonimide were added as crosslinking agents to 5 ml of the stimuli-sensitive magnetic nanocomposite dispersed in the PBS solution prepared in Example 1, 0.7 mg of Herceptin was added thereto, and then the resulting mixture was reacted at 4° C. After 6 hours, the mixture was centrifuged at 20,000 rpm for 45 minutes to separate unbound Herceptin and crosslinking agent. By the same method as described above, human IgG, instead of Herceptin, was added, thereby preparing a human IgG-binding stimuli-sensitive nanocomposite as a control group.

Amounts of Herceptin and IgG, which were bound to the nanocomposite, were indentified using a protein quantification kit.

### EXPERIMENTAL EXAMPLE 3

### Identification of Target specificity by Target Material

A targeting test of the target-specific stimuli-sensitive magnetic nanocomposite prepared in Example 2 was performed using an NIH3T6.7 cell overexpres sing a Herceptin receptor and an MDA-MB-231 cell low-expressing a Herceptin receptor.

After the target-specific stimuli-sensitive magnetic nanocomposite was treated to each of 5×10⁶ of NIH3T6.7 and MDA-MB-231 cells, the cells were incubated at 4° C. for 30 minutes and washed with PBS three times, and 400 μl of PBS was dispersion-added. To quantitatively analyze target specificity, a second target-specific ligand (goat anti-human IgG) to which fluorescein isothiocyanate (FITC) was bound was treated to each of the reacted cells, and incubated in a dark room at 4° C. for 20 minutes. Subsequently, target specificity was identified using FACScan (Beckton-Dickinson, Sunnyvale, Calif., USA) having CellQuest Software via flow cytometry and MRI.

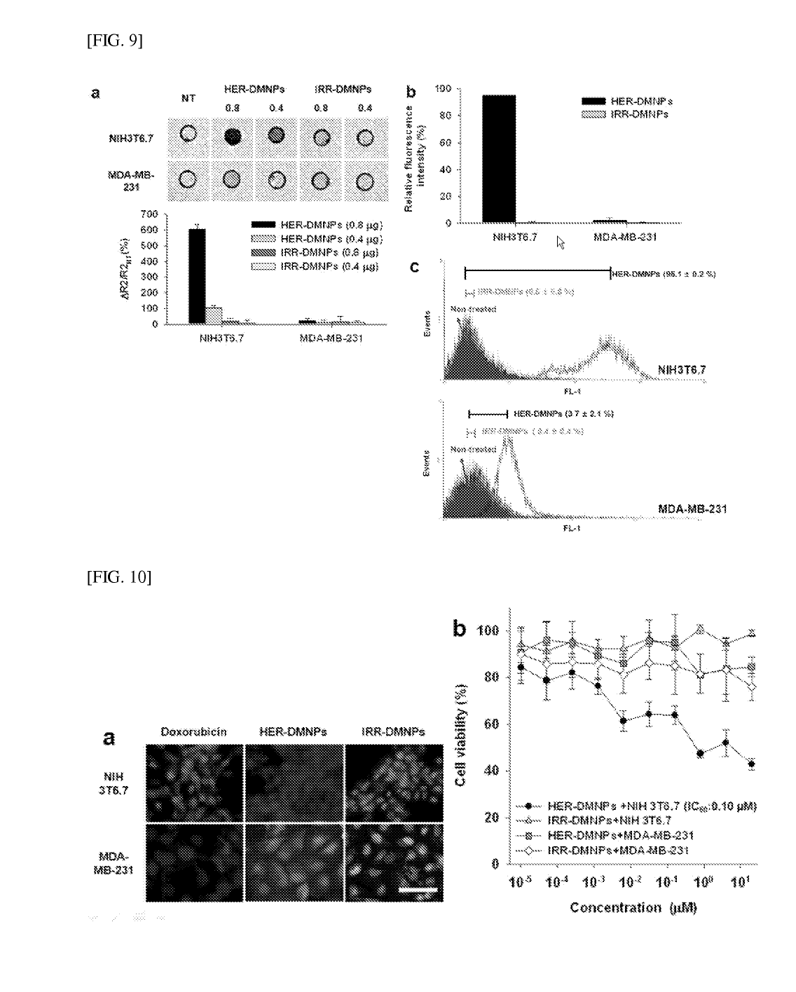

As shown in FIG. 9(A), it was observed that the target-specific stimuli-sensitive magnetic nanocomposite showed dark MRI and a high r2 value in a cell overexpressing a Herceptin receptor.

As shown in FIG. 9(B), it was identified that the target-specific stimuli-sensitive magnetic nanocomposite showed a high intensity of fluorescence of FITC in the cell overexpressing a Herceptin receptor, thereby proving target specificity to a specific cell

According to the results, it was identified that the target-specific stimuli-sensitive magnetic nanocomposite was effectively targeted.

### EXPERIMENTAL EXAMPLE 4

### Simultaneous Identification of Cell Targeting Ability and Drug Release of Target-Specific Stimuli-Sensitive Magnetic Nanocomposite

A test for simultaneously identifying a cell targeting ability and degree of drug release of the target-specific stimuli-sensitive magnetic nanocomposite prepared in Example 2 was performed.

For each of 12 wells, a cover glass was put on a bottom, 3×10⁵ of each of NIH3T6.7 and MDA-MB-231 cells were dispensed on the glass per well to incubate for 12 hours, and washed with PBS three times. After the washing with PBS, 0.05 mM of a Herceptin-binding target-specific stimuli-sensitive nanocomposite based on doxorubicin, an IgG-binding stimuli-sensitive nanocomposite as a control group, and doxorubicin were each added to the cells, and incubated for 30 minutes. After the cells were washed with PBS, a dye for a cell nucleus (Hoechst dye) was added to the cells, reacted for 10 minutes and washed with PBS three times, and then the cover glass was fixed to a slide glass to observe microscope images. The results are shown in FIG. 10(A).

Doxorubicin was identified from a nucleus of the doxorubicin-treated cell without distinction of cells, and only from a nucleus of the NIH3T6.7 cell of those treated with the Herceptin-binding target-specific stimuli-sensitive magnetic nanocomposite. From the control particle, doxorubicin was scarcely identified in either cell.

According to the above results, it was identified that the target-specific stimuli-sensitive magnetic nanocomposite was targeted, and whether the drug was or was not released.

### EXPERIMENTAL EXAMPLE 5

### Identification of Ability to Kill Cells of Target-Specific Stimuli-Sensitive Magnetic Nanocomposite

Using the Herceptin-binding target-specific stimuli-sensitive magnetic nanocomposite prepared in Example 2 and the human IgG-binding stimuli-sensitive magnetic nanocomposite as the control group, cytotoxicity with respect to each group of the NIH3T6.7 and MDA-MB-231 cells was evaluated by MTT assay, and the results are shown in FIG. 8(B). 5×10⁴ of each group of the cells was dispensed in each of 196 wells to incubate for 12 hours, and was treated with the Herceptin-binding target-specific stimuli-sensitive magnetic nanocomposite prepared in Example 2 and the human IgG-binding stimuli-sensitive magnetic nanocomposite as the control group with various concentrations. After 30-minute incubation, the cells were washed with PBS, and further incubated for 72 hours in a culture medium added. Afterward, cell viability was measured using a reagent for MTT assay.

Referring to FIG. 10(B), in the NIH3T6.7 cells treated with the target-specific stimuli-sensitive magnetic nanocomposite, cell viability was drastically decreased according to the increase in concentration, and a concentration having 50% cell viability (Inhibitory concentration: IC₅₀) was measured at 0.01 μM. However, under other conditions excluding the above, cell viability exceeded 80% at a concentration of 0.01 mM or less. Accordingly, cell death of a specific cancer cell caused by the target-specific stimuli-sensitive magnetic nanocomposite of Example 2 was identified.

### EXPERIMENTAL EXAMPLE 6

### Identification of Retention Behavior of Target-Specific Stimuli-Sensitive Magnetic Nanocomposite

To indentify maintenance of target diagnosis performance and behavior tendency of the target-specific stimuli-sensitive magnetic nanocomposite prepared in Example 2 using an animal model, first, the NIH3T6.7 cells were injected under the skin of a nude mouse to grow cancer for three days. Afterward, the nude mouse in which the cancer cell was generated was imaged in MRI (pre-injection), 0.2 ml of the target-specific stimuli-sensitive magnetic nanocomposite prepared in Example 2 was taken with a 1 ml syringe to inject into the tail of the imaged nude mouse, and then MRI was performed periodically. A test was subjected to the human IgG-binding stimuli-sensitive magnetic nanocomposite prepared in Example 2 as the control group in the same manner as described above.

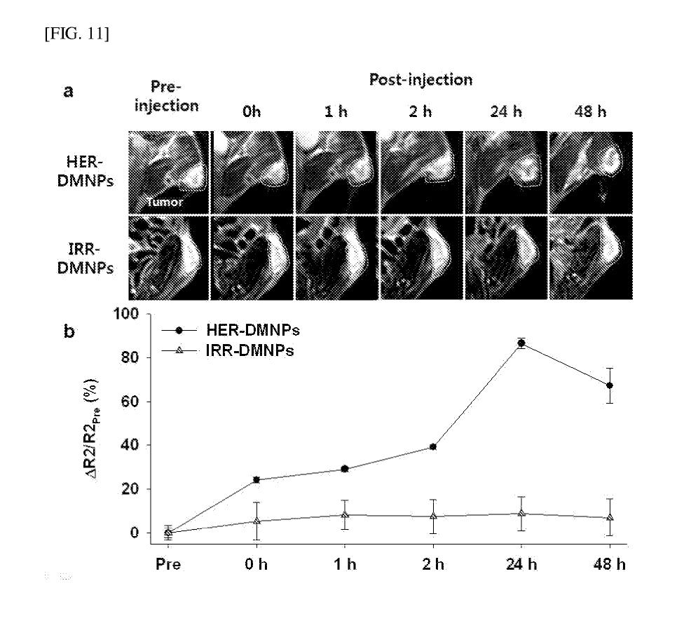

As a result, compared to peripheral tissues, an MRI image of a cancer lesion in the nude mouse into which the target-specific stimuli-sensitive magnetic nanocomposite was injected became darker according to the tendency to form blood vessels, which made distinct changes in images. Particularly, 24 hours after the injection, the image was the darkest. Accordingly, it was indentified that the composite showed excellent target specificity and was present in the mouse at this period of time. However, the control group did not show any significant difference between pre-injection and post-injection. The MRI results and relaxation graph are shown in FIGS. 11(A) and (B), respectively.

### EXPERIMENTAL EXAMPLE 7

### Identification of Diagnostic Effect of Target-Specific Stimuli-Sensitive Magnetic Nanocomposite

To identify a therapeutic effect of the target-specific stimuli-sensitive magnetic nanocomposite prepared in Example 2 using an animal model, first, the NIH3T6.7 cells were injected under the skin of a nude mouse to grow a cancer for three days. Afterward, the nude mouse in which the cancer cell was generated was imaged in MRI (pre-injection), 0.2 ml of the target-specific stimuli-sensitive magnetic nanocomposite prepared in Example 2 was taken with a 1 ml syringe, and then MRI was performed. Afterward, injection of the target-specific stimuli-sensitive magnetic nanocomposite was performed 4 times at three-day intervals (0, 3, 6 and 9 days), and MRI was performed on the first injection day (0 day), 24 hours after the first injection (1 day), 3 days after the final injection (12 days), and 4 days after the final injection (13 days). A test was subjected to the human IgG-binding stimuli-sensitive magnetic nanocomposite prepared in Example 2 as the control group in the same manner as described above.

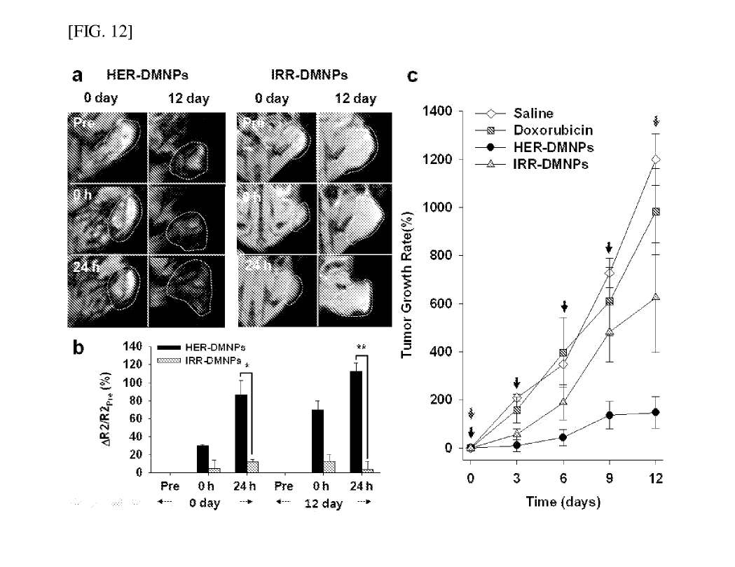

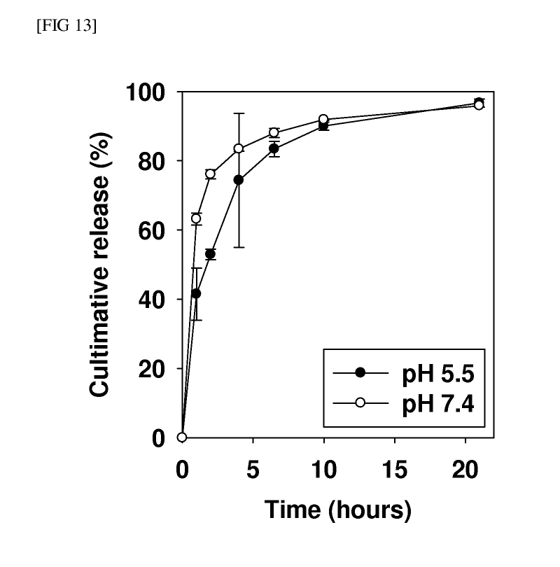

As a result, compared to peripheral tissues, an MRI image of a cancer lesion in the nude mouse into which the target-specific stimuli-sensitive magnetic nanocomposite was injected was darker even 24 hours after the injection. Accordingly, it was identified that the composite had an ability to diagnose a tumor. However, the control group did not show any significant difference between pre-injection and post-injection. The MRI results and relaxation graph are shown in FIGS. 12(A) and (B), respectively.

### EXPERIMENTAL EXAMPLE 8

### Identification of Therapeutic Effect of Target-Specific Stimuli-Sensitive Magnetic Nanocomposite

The target-specific stimuli-sensitive magnetic nanocomposite was injected into a nude mouse into which a cancer cell was injected in the same manner as described in Experimental Example 6 to identify a therapeutic effect.

A size and volume of the lesion were measured on the days of injecting the target-specific stimuli-sensitive magnetic nanocomposite and the last day of performing MRI (0, 3, 6, 9 and 12 days), and the measurement was subjected to each of three control groups injected with saline, doxorubicin and the human IgG-binding stimuli-sensitive magnetic nanocomposite in the same manner as described above to identify a target-specific therapeutic effect.

The nude mouse into which the target-specific stimuli-sensitive magnetic nanocomposite was injected showed a significantly lower cancer growth rate (%) than other groups, and the results are shown in FIG. 12(C).

As magnetic nanoparticles and a pharmaceutically active ingredient are coated with an amphiphilic compound to which a pyrene structure is bound, a stimuli-sensitive magnetic nanocomposite of the present invention has stability in an aqueous solution, excellent magnetic properties, and shows a change in drug release behavior due to a specific stimulus. In addition, as the magnetic nanocomposite is bound with a targetable factor, it can exhibit excellent performance as a composition for simultaneously diagnosing and treating a specific disease.

While the invention has been shown and described with reference to certain exemplary embodiments thereof, it will be understood by those skilled in the art that various changes in form and details may be made therein without departing from the scope of the invention as defined by the appended claims.

1. A stimuli-sensitive magnetic nanocomposite, comprising:
a core containing at least one magnetic nanoparticle; and
a shell containing an amphiphilic compound having at least one hydrophobic domain and at least one hydrophilic domain,
wherein the hydrophobic domain includes a material including a pyrene structure to which a pharmaceutically active ingredient is chemically bound.

2. The nanocomposite according to claim 1, wherein the nanoparticle is a metal, magnetic material or magnetic alloy.

3. The nanocomposite according to claim 2, wherein the magnetic material is at least one selected from the group consisting of Co, Mn, Fe, Ni, Gd, Mo, MM′₂O₄ and MₓO_{y} (M and M′ are each independently Co, Fe, Ni, Mn, Zn, Gd or Cr, and x and y satisfy “0<x≦3” and “0<y≦5”).

4. The nanocomposite according to claim 1, wherein the magnetic nanoparticle is bound with an organic surface stabilizer.

5. The nanocomposite according to claim 1, wherein the hydrophobic domain is a hydrophobic compound to which a material having a pyrene structure is bound.

6. The nanocomposite according to claim 5, wherein the hydrophobic compound is a saturated fatty acid, unsaturated fatty acid or hydrophobic polymer.

7. The nanocomposite according to claim 6, wherein the hydrophobic polymer is at least one selected from the group consisting of polyphosphazene, polylactide, polylactide-co-glycolide, polycaprolactone, polyanhydride, polymaleic acid or a derivative thereof, polyalkylcyanoacrylate, polyhydroxybutyrate, polycarbonate, polyorthoester, a hydrophobic polyamino acid, and a hydrophobic vinly-based polymer.

8. The nanocomposite according to claim 1, wherein the material including a pyrene structure is at least one selected from the group consisting of pyrene, pyrenebutyric acid, pyrene methylamine, 1-aminopyrene, pyrene-1-boronic acid and an organic molecule including a pyrene structure.

9. The nanocomposite according to claim 1, wherein the hydrophilic domain is at least one selected from the group consisting of polyalkyleneglycol (PAG), polyetherimide (PEI), polyvinylpyrrolidone (PVP), hydrophilic polyamino acid (PAA), a hydrophilic vinyl-based polymer, a hydrophilic acryl-based polymer, and a polysaccharide-based polymer.

10. The nanocomposite according to claim 1, wherein the pharmaceutically active ingredient is at least one selected from the group consisting of an anticancer agent, an antibiotic, a hormone, a hormone antagonist, an interleukin, an interferon, a growth factor, a tumor necrosis factor, an endotoxin, a lymphotoxin, a urokinase, a streptokinase, a tissue plasminogen activator, a protease inhibitor, an alkylphosphocholine, a component marked with a radio isotope, a cardiovascular drug, a gastrointestinal drug, and a neural drug.

11. The nanocomposite according to claim 1, wherein the hydrophilic domain is bound with a tissue-specific binding component.

12. The nanocomposite according to claim 11, wherein the tissue-specific binding component is at least one selected from the group consisting of an antigen, an antibody, RNA, DNA, hapten, avidin, streptavidin, neutravidin, protein A, protein G, lectin, selectin, a radio isotope-marked component, and a material capable of specifically binding to a tumor marker.

13. A method of preparing a stimuli-sensitive magnetic nanocomposite, comprising:
mixing magnetic nanoparticles, an amphiphilic compound having at least one hydrophilic domain and at least one hydrophobic domain including a material including a pyrene structure to which a pharmaceutically active ingredient is chemically bound, and a pharmaceutically active ingredient.

14. The method according to claim 13, wherein the magnetic nanoparticles are prepared by mixing a precursor of the nanoparticles with an organic surface stabilizer in the presence of a solvent and heating the mixture to thermally decompose the precursor of the nanoparticles.

15. The method according to claim 13, wherein the amphiphilic compound is prepared by binding a hydrophilic compound with a hydrophobic compound having a material including a pyrene structure using a crosslinking agent.

16. The method according to claim 13, further comprising:
dissolving magnetic nanoparticles in an organic solvent to prepare a first oil phase;
dissolving an amphiphilic compound and a pharmaceutically active ingredient in an organic solvent to prepare a second oil phase;
mixing the first oil phase, the second oil phase and an aqueous phase to form an emulsion; and
evaporating the oil phases from the emulsion.

17. The method according to claim 13, further comprising:
binding a tissue-specific binding component to the nanocomposite.

18. A contrast composition for simultaneous diagnosis and treatment, comprising:
the stimuli-sensitive magnetic nanocomposite according to claim 1 as an effective ingredient.

19. A pharmaceutical composition, comprising:
the stimuli-sensitive magnetic nanocomposite according to claim 1 as an effective ingredient.

20. A multi-diagnostic probe, comprising:
the stimuli-sensitive magnetic nanocomposite according to claim 1; and
a diagnostic probe.
